(19) United States
(12) Patent Application Publication
(10) Pub. No.: US 2026/0315716 A1
(43) Pub. Date: Oct. 15, 2026

(54) BITE GUARD

(71) Applicant: Obsolete Engineering Co., Washington, DC (US)

(72) Inventor: Amir M. TAHA, Washington, DC (US)

(21) Appl. No.: 19/467,320

(22) Filed: Feb. 2, 2026

Related U.S. Application Data
(60) Provisional application No. 63/752,961, filed on Feb. 3, 2025.

(51) Int. Cl. A61M 16/04; A61C 7/08; A61F 5/56

(52) U.S. Cl. CPC A61M 16/0493; A61C 7/08; A61F 5/566

(57) ABSTRACT

A bite guard configured for insertion into an oral cavity of a patient includes a first bite block and a second bite block spaced apart laterally and each including an upper surface configured to engage upper teeth of the patient and a lower surface configured to engage lower teeth of the patient, each of the first and second bite blocks having an anterior end and a posterior end. A bridge member joins the first and second bite blocks at medial sides thereof, upper barriers extend upwardly from the medial sides of the first and second bite blocks, and lower barriers extend downwardly from the medial sides of the first and second bite blocks. The upper barriers and the bridge member define an opening providing access to the oral cavity, and the lower barriers and the bridge member define a cavity configured to receive a tongue of the patient.

### CROSS-REFERENCE TO RELATED APPLICATIONS

This application claims the benefit of U.S. provisional application Serial No. 63/752,961 filed Feb. 3, 2025, the disclosure of which is hereby incorporated in its entirety by reference herein.

### TECHNICAL FIELD

Embodiments relate to a bite guard configured for insertion into an oral cavity of patient suffering from certain medical conditions or undergoing various medical procedures.

### BACKGROUND

Bite guards are designed to be placed in a patient’s mouth between the upper and lower teeth, and may be used to protect the teeth, tongue, and mouth of patients suffering from various medical conditions. For example, a bite guard may be used when treating a patient experiencing a seizure to prevent damage to their teeth and tongue as their jaw is clenched with considerable force. In another example, a bite guard can also be used for patients who suffer from brain damage experience neurogenic bruxism, which involves involuntarily biting down at maximum strength.

Additionally, bite guards are helpful for preventing trauma to a patient’s teeth and tongue due to tubes, catheters, scopes, or other medical equipment which must be inserted in the patient's mouth used during certain medical procedures. As well as protecting the oral structures of the patient, bite guards prevent damage to the medical equipment itself. For example, a bite guard can avoid deformation of tubes and catheters due to contact by a patient’s teeth, which could compromise the ability of these conduits to deliver or remove air or fluids. Sensitive equipment such as endoscopes may also be safeguarded from the patient’s teeth by use of a bite guard.

### SUMMARY

In one or more embodiments, a bite guard configured for insertion into an oral cavity of a patient includes a first bite block and a second bite block spaced apart laterally, the first bite block and the second bite block each including an upper surface configured to engage upper teeth of the patient and a lower surface configured to engage lower teeth of the patient, each of the first and second bite blocks having an anterior end and a posterior end. A bridge member joins the first bite block and the second bite block at medial sides thereof, upper barriers extending upwardly from the medial sides of the first and second bite blocks, and lower barriers extending downwardly from the medial sides of the first and second bite blocks. The upper barriers and a superior side of the bridge member define an opening providing access to the oral cavity, and the lower barriers and an inferior side of the bridge member define a cavity configured to receive a tongue of the patient.

In one or more embodiments, a distance between the anterior ends is smaller than a distance between the posterior ends. In one or more embodiments, the anterior end of each of the first and second bite blocks is thicker than the posterior end of each of the first and second bite blocks. In one or more embodiments, at least one of the first and second bite blocks includes a recessed portion formed in the upper surface adjacent to the anterior end and the upper barriers. In one or more embodiments, the first bite block and the second bite block each have a generally rectangular body surrounded by a smooth, rounded outer surface. In one or more embodiments, the upper surface and the lower surface of each of the first and second bite blocks include a plurality of raised ridges.

In one or more embodiments, the upper barriers and the lower barriers each have a generally planar configuration with rounded edges. In one or more embodiments, the lower barriers have a greater height dimension than the upper barriers.

In one or more embodiments, the bridge member has an arched configuration including a central portion extending upwardly relative to end portions connected to the first and second bite blocks. In one or more embodiments, at least one of the bridge member, the first bite block, and the second bite block includes orientation indicia thereon.

In one or more embodiments, the bite guard includes arms extending from the anterior ends of the first and second bite blocks. In one or more embodiments, the arms each include a proximal section, a curved section extending from the proximal section in a posterior direction, and a distal section extending laterally from the curved section. In one or more embodiments, the arms each include an attachment portion configured to receive a retaining strap.

In one or more embodiments, a bite guard configured for insertion into an oral cavity of a patient includes a first bite block and a second bite block spaced apart laterally, the first bite block and the second bite block each including an upper surface configured to engage upper teeth of the patient and a lower surface configured to engage lower teeth of the patient, each of the first and second bite blocks having an anterior end and a posterior end. A bridge member joins the first bite block and the second bite block at medial sides thereof. Upper barriers extending upwardly from the medial sides of the first and second bite blocks, and lower barriers extending downwardly from the medial sides of the first and second bite blocks, wherein the upper barriers and the bridge member define an opening providing access to the oral cavity, and the lower barriers and the bridge member define a cavity configured to receive a tongue of the patient. Arms extend from the anterior ends of the first and second bite blocks, each arm including an attachment portion. A strap is removably secured to the attachment portions, wherein the arms and the strap are configured to provide extraoral stabilization of the bite guard. In one or more embodiments, the strap is adjustable.

In one or more embodiments, a bite guard configured for insertion into an oral cavity of a patient includes a generally U-shaped upper member having an upper surface configured to engage upper teeth of the patient, the upper member having spaced upper side portions with an upper anterior end portion therebetween. A generally U-shaped lower member is spaced from the upper member and has a lower surface configured to engage lower teeth of the patient, the lower member having spaced lower side portions with a lower anterior end portion therebetween. A support structure joins the upper side portions with the lower side portions, the support structure including a first support member and a second support member, wherein an opening is defined between the upper anterior end portion and the lower anterior end portion and extending through the bite guard between the first support member and the second support member for providing access into the oral cavity

In one or more embodiments, the upper member includes an upper lip barrier extending upwardly from the upper anterior end portion, and the lower member includes a lower lip barrier extending downwardly from the lower anterior end portion. In one or more embodiments, the upper lip barrier has a generally curved shape symmetric about a central longitudinal axis and includes an indentation configured to accommodate a maxillary frenum of the patient. In one or more embodiments, the lower member further includes a ledge extending downwardly from a posterior edge of the lower anterior end portion, the ledge configured to latch onto mandibular incisors of the patient. In one or more embodiments, the upper member includes upper medial lips extending upwardly from inner edges of side portions of the upper member, and the lower member includes lower medial lips extending downwardly from inner edges of side portions of the lower member, the upper medial lips and the lower medial lips configured to inhibit a tongue of the patient from contacting the upper surface and the lower surface, respectively.

### BRIEF DESCRIPTION OF THE DRAWINGS

FIG. 1 is a top perspective view of a bite guard according to one or more embodiments;

FIG. 2 is a front perspective view of the bite guard of FIG. 1;

FIG. 3 is a bottom perspective view of the bite guard of FIG. 1;

FIG. 4 is a front view of the bite guard of FIG. 1;

FIG. 5 is a top view of the bite guard of FIG. 1;

FIG. 6 is a left side view of the bite guard of FIG. 1;

FIG. 7 is a rear perspective view of the bite guard of FIG. 1;

FIG. 8 is a top view of a bite guard according to one or more embodiments;

FIG. 9 is a cross-sectional view of the bite guard of FIG. 8 taken along line A-A according to one or more embodiments;

FIG. 10 is a front view of a bite guard according to one or more embodiments;

FIG. 11 is a rear view of a bite guard according to one or more embodiments;

FIG. 12 is a cross-sectional view of the bite guard of FIG. 11 taken along line B-B according to one or more embodiments;

FIG. 13 is a left side view of a bite guard according to one or more embodiments;

FIG. 14 is a top perspective view of another bite guard according to one or more embodiments;

FIG. 15 is a side perspective view of the bite guard of FIG. 14;

FIG. 16 is a bottom perspective view of the bite guard of FIG. 14;

FIG. 17 is a left side view of the bite guard of FIG. 14;

FIG. 18 is a rear view of the bite guard of FIG. 14;

FIG. 19 is a front view of the bite guard of FIG. 14;

FIG. 20 is a top view of the bite guard of FIG. 14;

FIG. 21 is a bottom view of the bite guard of FIG. 14;

FIG. 22 is a top perspective view of another bite guard according to one or more embodiments;

FIG. 23 is a front view of the bite guard of FIG. 22;

FIG. 24 is a rear view of the bite guard of FIG. 22;

FIG. 25 is a top view of the bite guard of FIG. 22;

FIG. 26 is a bottom view of the bite guard of FIG. 22;

FIG. 27 is a right side view of the bite guard of FIG. 22;

FIG. 28 is a left side view of the bite guard of FIG. 22; and

FIG. 29 is a top perspective view of the bite guard of FIG. 22 with an exemplary strap attached thereto.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

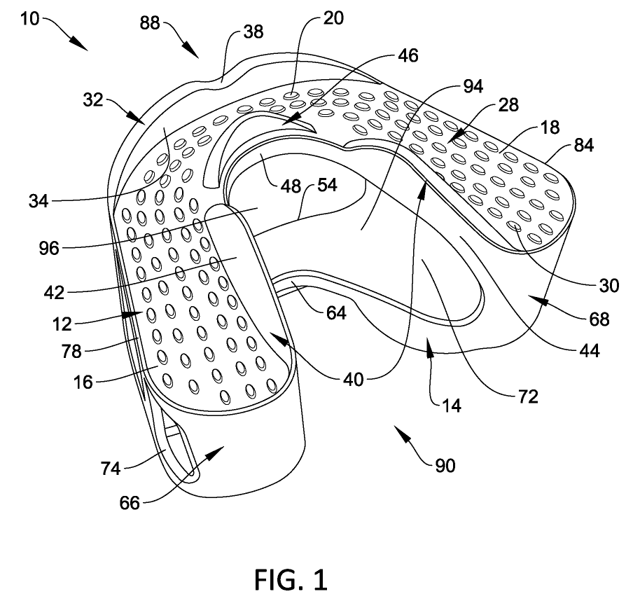

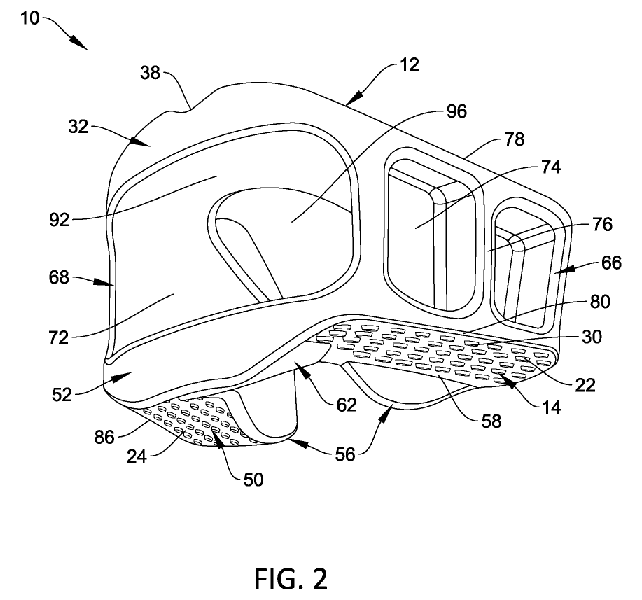

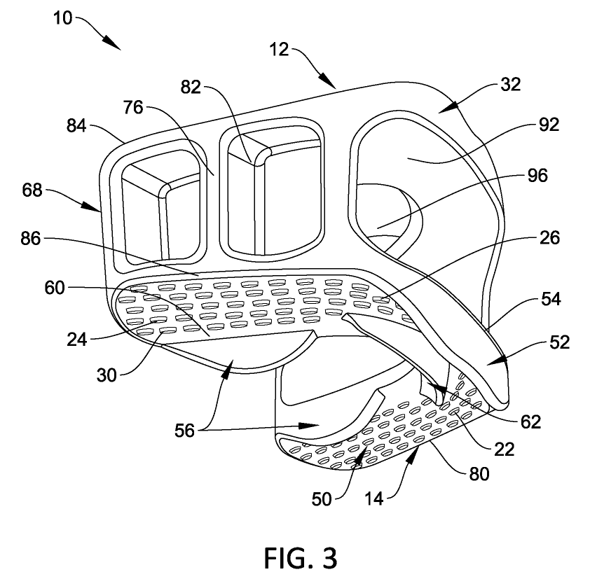

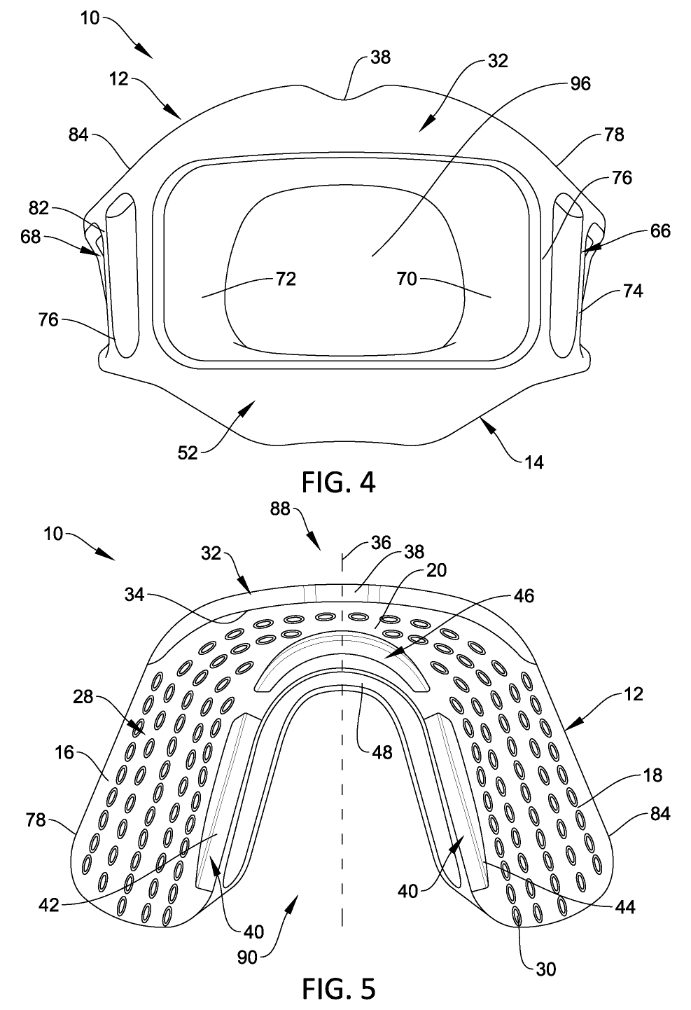

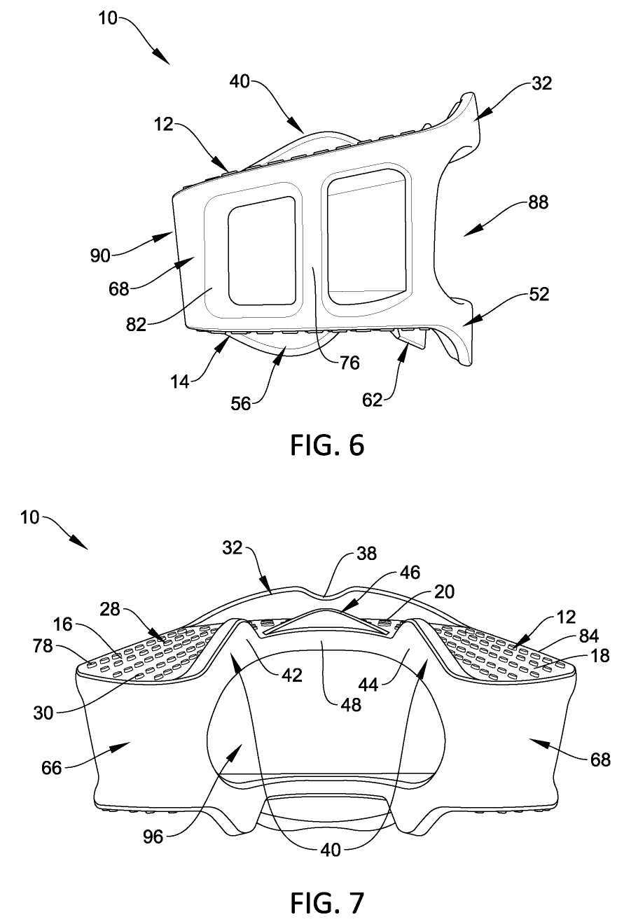

With reference first to FIGS. 1-7, various views of a bite guard 10 according to one or more embodiments are illustrated. The bite guard 10 is configured for insertion into an oral cavity of a patient suffering from certain medical conditions or undergoing various medical procedures. The bite guard 10 may be designed to be securely retained against the upper teeth and the lower teeth to prevent accidental displacement during intubation or other procedures. This secure retention ensures that proper positioning of the bite guard 10 is maintained so that it can properly protect the patient’s teeth, tongue, and other oral structures.

According to one or more embodiments, the bite guard 10 maintains the patient’s mouth in an open position under heavy biting forces, providing continued access to the oral cavity and protecting the oral structures from injury. For example, the bite guard 10 protects the anterior teeth of the patient from lateral forces during trans-esophageal procedures and any other maneuvers surrounding the oral cavity during surgery or in-patient care, and protects the tongue from trauma related to occlusal forces. While performing a head tilt, the bite guard 10 is configured to perform a continuous mandibular chin thrust of the patient by effectively locking the bottom teeth in place relative to the maxilla prior to the maneuver. This elevates the soft palate and surrounding soft tissue structures in the head and neck to prevent oxygen desaturation during anesthesia, resulting in a lower incidence of postoperative hypoxemia. Furthermore, the bite guard 10 isolates the entrance of the patient’s oral cavity to aid in pathway guidance down the throat during intubation, emergencies, and other trans-esophageal procedures.

The bite guard 10 includes an upper member 12 spaced from a lower member 14. In one or more embodiments, the upper member 12 and the lower member 14 may each have a generally U-shaped configuration, so as to correspond with the anatomical configuration of the upper teeth and lower teeth of a patient, respectively. In particular, the upper member 12 may have upper side portions including a first side portion 16 spaced from a second side portion 18, with the first side portion 16 and the second side portion 18 joined by an upper anterior end portion 20. Likewise, the lower member 14 may have lower side portions including a first side portion 22 spaced from a second side portion 24, with the first side portion 22 and the second side portion 24 joined by a lower anterior end portion 26.

The upper member 12 includes an upper surface 28 configured to engage the upper teeth of a patient when the bite guard 10 is positioned in the oral cavity. In one or more embodiments, the upper surface 28 may include a plurality of raised nodes 30 configured to provide a grip surface to prevent sliding of the upper member 12 along the upper teeth of the patient. The upper member 12 may include an upper lip barrier 32 extending upwardly from a front edge 34 of the upper anterior end portion 20 which is configured to protect the upper teeth of a patient from damage, such as during intubation or the entry of tools into the oral cavity. The upper lip barrier 32 may have a generally curved shape symmetric about a central longitudinal axis 36 of the bite guard 10, wherein the upper lip barrier 32 may include an indentation 38 adjacent to the central longitudinal axis 36 to accommodate the maxillary frenum of the patient.

The upper member 12 may further include upper medial lips 40 extending upwardly from each of the inner edge 42 of the first side portion 16 and the inner edge 44 of the second side portion 18. The upper medial lips 40 may have a generally curved configuration and function to protect the patient’s tongue by preventing the tongue from lying over the upper surface 28 of the bite guard 10 and receiving trauma from the maxillary molars during use. The upper member 12 may further include a support wall 46 extending upwardly from a back edge 48 of the upper anterior end portion 20 which may serve to help maintain the integrity of the bite guard 10 when the patient bites down, preventing the bite guard 10 from collapsing and deforming under axial stress. The support wall 46 may have a curved profile with a generally convex anterior shape, thus aiding in comfort, positioning, and retention of the upper teeth of the patient between the upper lip barrier 32 and the support wall 46 when the bite guard 10 is in use.

Correspondingly, the lower member 14 includes a lower surface 50 configured to engage the lower teeth of a patient when the bite guard 10 is positioned in the oral cavity. In one or more embodiments, the lower surface 50 may include a plurality of raised nodes 30 configured to provide a grip surface to prevent sliding of the lower member 14 along the lower teeth of the patient. The lower member 14 may include a lower lip barrier 52 extending downwardly from a front edge 54 of the lower anterior end portion 26 which is configured to protect the lower teeth of a patient from damage, such as during intubation or the entry of tools into the oral cavity. The lower lip barrier 52 also functions as a major support structure to prevent deformation of the bite guard 10 under high biting forces (e.g. up to 200 N/cm). The lower lip barrier 52 may have a generally curved shape symmetric about the central longitudinal axis 36 of the bite guard 10.

As with the upper member 12, the lower member 14 may further include lower medial lips 56 extending downwardly from each of the inner edge 58 of the first side portion 22 and the inner edge 60 of the second side portion 24. The lower medial lips 56 may have a generally curved configuration and function to protect the patient’s tongue by preventing the tongue from lying under the lower surface 50 of the bite guard 10 and receiving trauma from the mandibular molars during use. The lower member 14 may further include a ledge 62 extending downwardly from a back edge 64 of the lower anterior end portion 26. The ledge 62 may have a curved profile with a generally convex anterior shape, thus aiding in comfort, positioning, and retention of the lower teeth of the patient between the lip barrier 52 and the ledge 62 when the bite guard 10 is in use. The ledge 62 may also serve to latch onto the mandibular incisors of the patient, locking the mandible in place relative to the maxilla. By locking the mandible in place relative to the cranium via the bite guard 10, the mandible is protruded anteriorly as the patient performs a superior head tilt maneuver. This lifts the tongue away from the posterior pharynx and improves airway patency.

The upper member 12 and the lower member 14 are joined in spaced relationship by a support structure including a first support member 66 disposed between the first side portion 16 of the upper member 12 and the first side portion 22 of the lower member 14, and by a second support member 68 disposed between the second side portion 18 of the upper member 12 and the second side portion 24 of the lower member 14. In one or more embodiments, the upper member 12, the lower member 14, the first support member 66, and the second support member 68 are integrally formed, although other constructions are also contemplated.

The first support member 66 has an inner surface 70 extending generally perpendicularly between the upper member 12 and the lower member 14, wherein the inner surface 70 may be outboard of the inner edge 42 of the first side portion 16 of the upper member 12 and the inner edge 58 of the first side portion 22 of the lower member 14. Likewise, the second support member 68 has an inner surface 72 extending generally perpendicularly between the upper member 12 and the lower member 14, wherein the inner surface 72 may be outboard of the inner edge 44 of the second side portion 18 of the upper member 12 and the inner edge 60 of the second side portion 24 of the lower member 14.

The first support member 66 has an outer surface 74 defined by a plurality of generally vertical ribs or struts 76 extending between the upper member 12 and the lower member 14, wherein the outer surface 74 may be aligned with an outer edge 78 of the first side portion 16 of the upper member 12 and with an outer edge 80 of the first side portion 22 of the lower member 14. Correspondingly, the second support member 68 has an outer surface 82 defined by a plurality of generally vertical ribs or struts 76 extending between the upper member 12 and the lower member 14, wherein the outer surface 82 may be aligned with an outer edge 84 of the second side portion 18 of the upper member 12 and with an outer edge 86 of the second side portion 24 of the lower member 14. In one or more embodiments, the first support member 66 and the second support member 68 each widen from an anterior end 88 of the bite guard 10 to a posterior end 90 of the bite guard 10, such that the struts 76 may have different depths along the first support member 66 and the second support member 68.

The first support member 66 and the second support member 68 are designed to prevent the bite guard 10 from collapsing when experiencing axial stress exerted from the back molar teeth, and the wider struts 76 at the posterior end 90 may provide additional support for the large biting forces in that area. While the first support member 66 and the second support member 68 could alternatively have a solid construction and uniform cross-section between the inner surface 70, 72 and the outer surface 74, 82 of each support member 66, 68, use of the struts 76 provides the requisite strength while reducing the weight and materials required for the bite guard 10.

The inner surface 70 of the first support member 66, the inner surface 72 of the second support member 68, a lower surface 92 of the upper member 12, and an upper surface 94 of the lower member 14 define an opening 96 through the bite guard 10 from the anterior end 88 to the posterior end 90 of the bite guard 10 which provides access into the patient’s oral cavity. The opening 96 is defined between the upper anterior end portion 20 and the lower anterior end portion 26, and extends through the bite guard 10 between the first support member 66 and the second support member 68. At the anterior end 88, the opening 96 may extend along the entire upper anterior end portion 20 and entire lower anterior end portion 26, from the first support member 66 to the second support member 68. In one or more embodiments, the inner surface 70, the inner surface 72, the lower surface 92, and the upper surface 94 are all smooth with rounded transitions therebetween to provide a comfortable, safe cavity for the patient’s tongue and also for ease of placement of tubes, scopes, and other equipment.

The opening 96 creates a guiding pathway for easy and reliable insertion of an endotracheal tube or other catheter, scope, or medical equipment through the bite guard 10 and into the oral cavity. With its large opening 96, the bite guard 10 provides for safe passage of such devices without causing damage to the patient’s oral structures while allowing wide angle visibility for medical professionals during these procedures. The width of the opening 96 also allows flexibility in positioning and adjusting tubes, scopes, and the like within the patient's mouth.

In one or more embodiments, the bite guard 10 may be constructed from thermoplastic elastomers, plastics, silicones and any other similar bio-compatible materials, and may be constructed to be disposable or reusable and sterilizable. The material used for the bite guard 10 should be strong, rigid, and resilient enough to maintain structural integrity under the biting force of the patient and absorb the shock thereof, yet soft and compliant enough so as to be comfortable to wear and not cause damage to the gums, teeth, and oral cavity. In one or more embodiments, a weight of the bite guard 10 may be approximately 15 g.

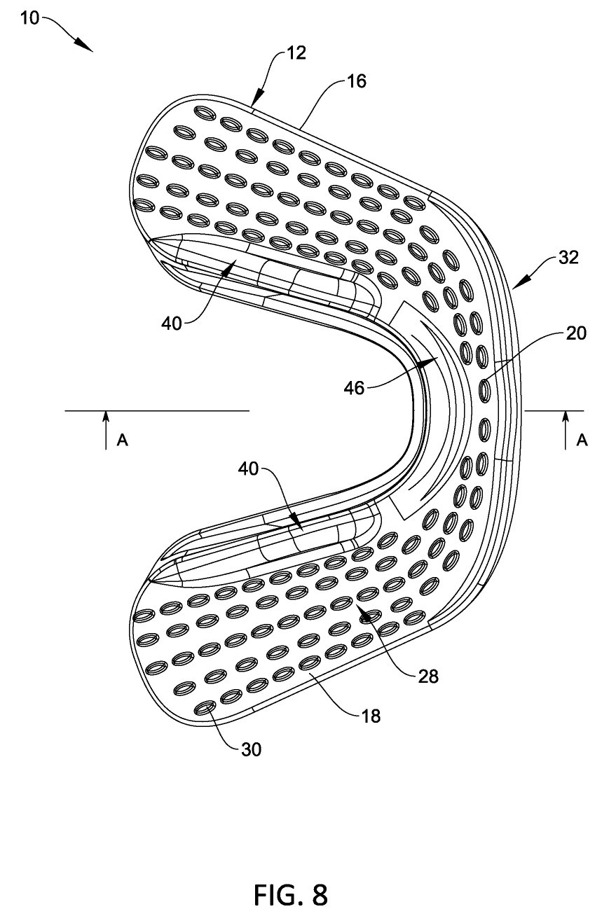

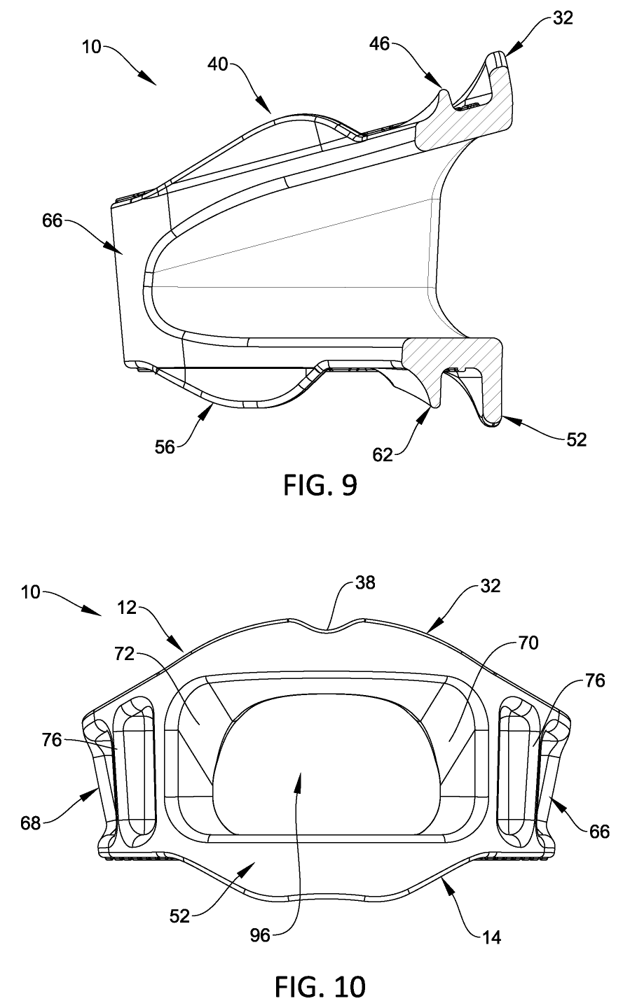

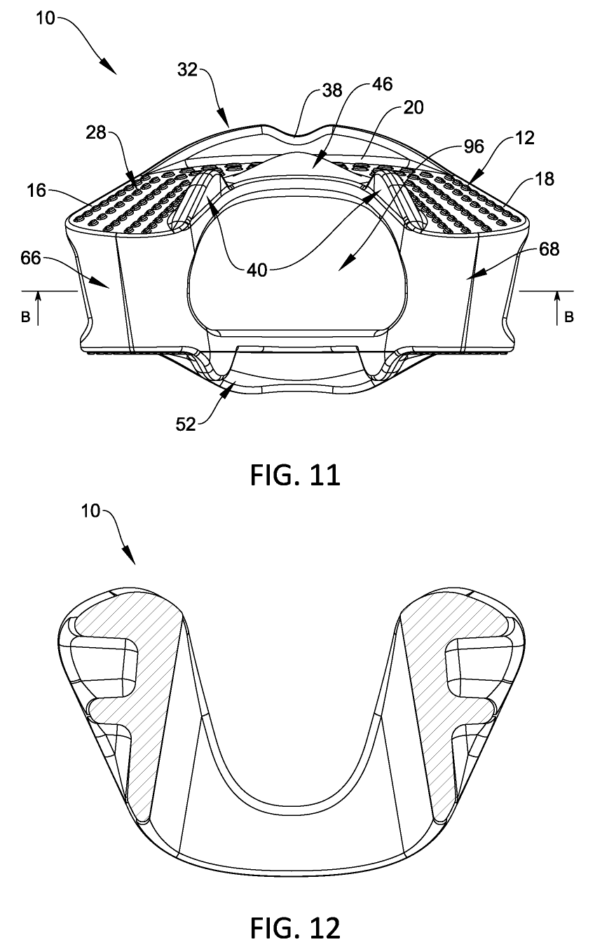

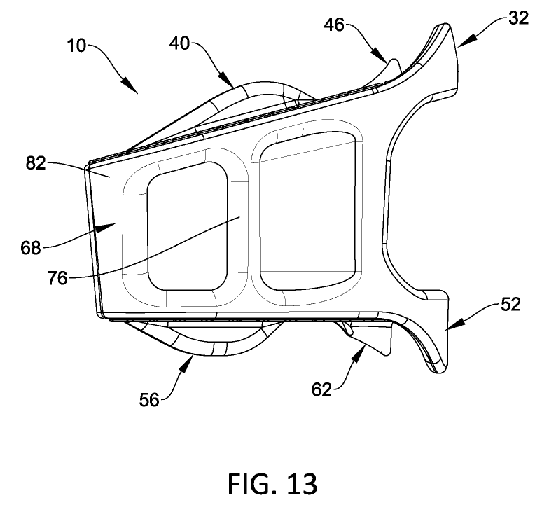

With reference to FIGS. 8-13, various views of the bite guard 10 are illustrated. The bite guard 10 may have a scalable design, may be available in a range of different sizes, or a have custom design for each patient. In one or more embodiments, the bite guard 10 is symmetric about the central longitudinal axis 36.

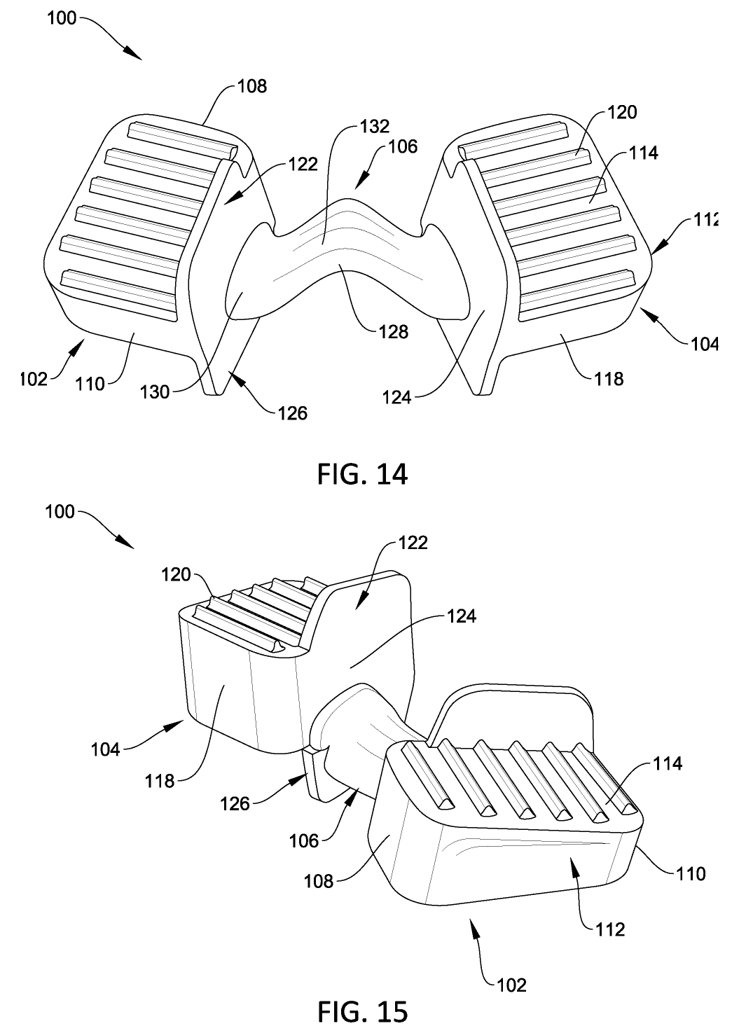

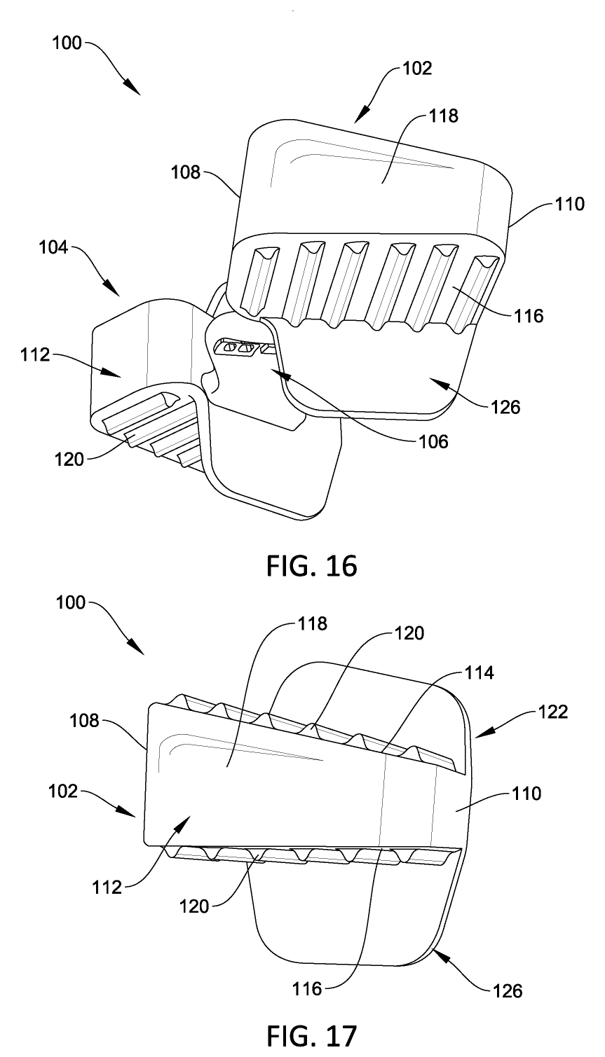

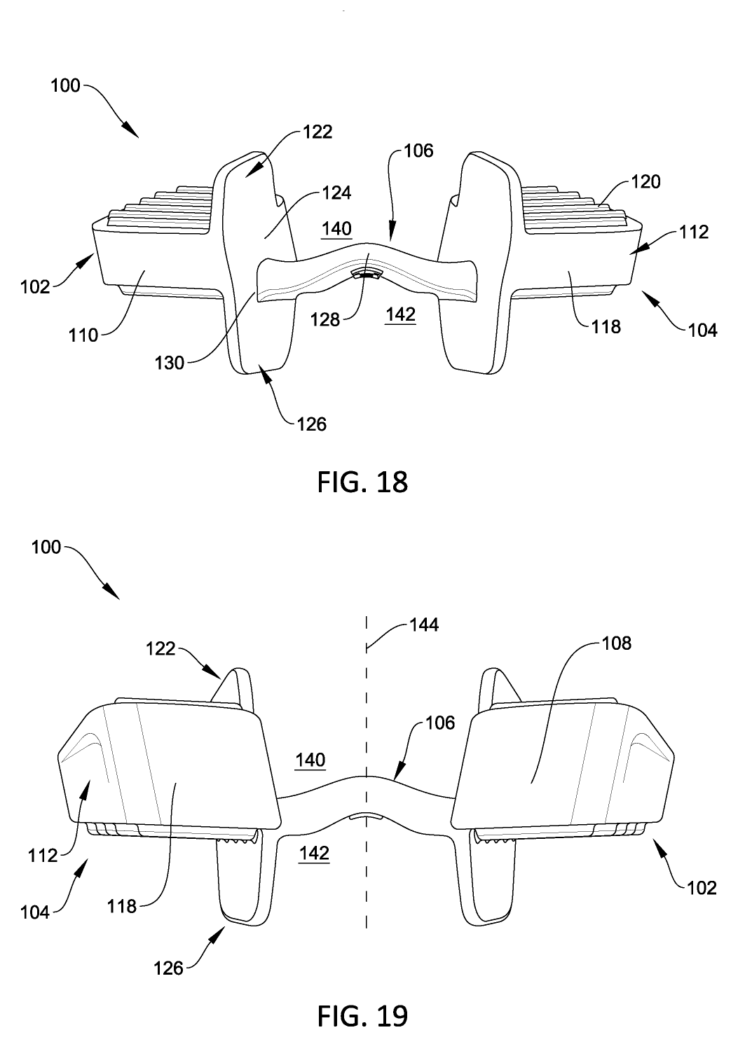

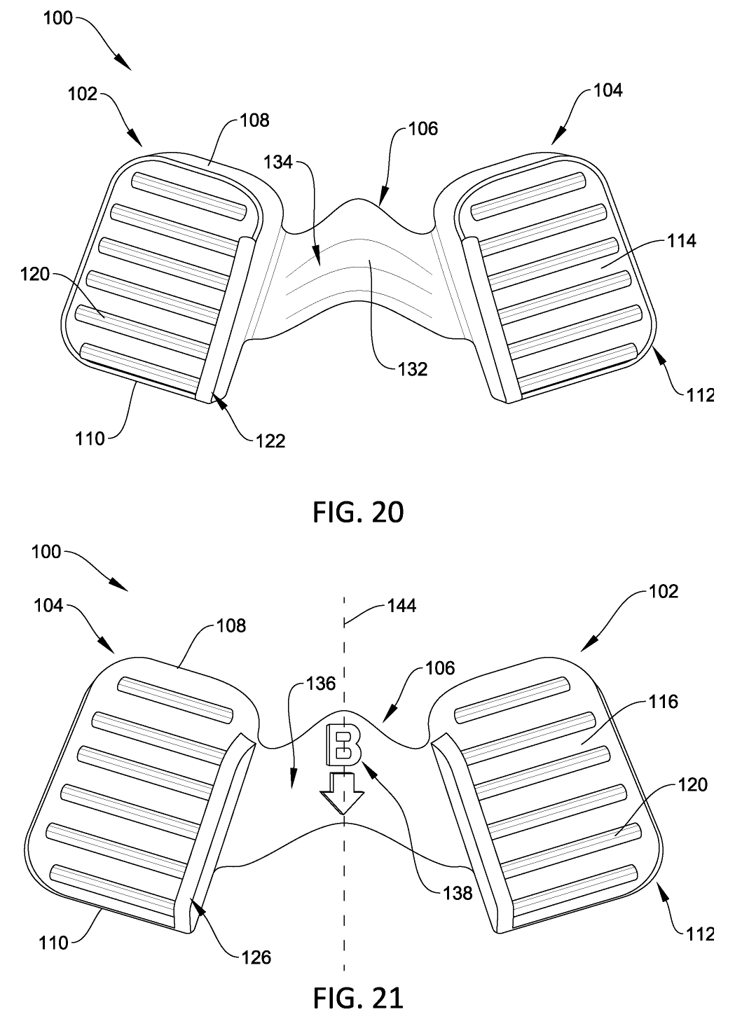

Turning now to FIGS. 14-21, various views of another bite guard 100 according to one or more embodiments are illustrated. It is understood that the features shown and described herein for bite guard 10 may be applicable to bite guard 100 and vice versa.

As with bite guard 10, bite guard 100 is configured for insertion into an oral cavity of a patient suffering from certain medical conditions or undergoing various medical procedures. The bite guard 100 may be designed to be securely retained against the upper teeth and the lower teeth to prevent accidental displacement during intubation or other procedures. This secure retention ensures that proper positioning of the bite guard 100 is maintained so that it can properly protect the patient’s teeth, tongue, and other oral structures. According to one or more embodiments, the bite guard 100 maintains the patient’s mouth in an open position under heavy biting forces, providing continued access to the oral cavity and protecting the oral structures from injury during use.

The bite guard 100 may have a bilateral configuration including a first bite block 102 spaced from a second bite block 104, wherein the first bite block 102 and the second bite block 104 are joined by a bridge member 106. In one or more embodiments, the first bite block 102, the second bite block 104, and the bridge member 106 are integrally formed, although other constructions are also contemplated. As illustrated, the first bite block 102 and the second bite block 104 may be oriented such that a distance between the anterior ends 108 of the first and second bite blocks 102, 104 is smaller than a distance between the posterior ends 110 of the first and second bite blocks 102, 104. Such a configuration corresponds to an expansion of a width of the lower jaw bone in the anterior to posterior direction, providing an ergonomic and comfortable fit of the bite guard 100 in a patient’s oral cavity.

In one or more embodiments, the first bite block 102 and the second bite block 104 may each have a generally rectangular body 112 with an upper surface 114 and a lower surface 116 surrounded by an outer surface 118. The outer surfaces 118 of the first bite block 102 and the second bite block 104 may be generally smooth and rounded so as to facilitate comfort for the patient as the bite guard 100 engages parts of the oral cavity during use. The body 112 of each of the first and second bite blocks 102, 104 may each have a thicker anterior end 108 compared with the posterior end 110, corresponding to a decreasing distance between the upper and lower teeth of a patient in an anterior to posterior direction, again facilitating an ergonomic and comfortable fit of the bite guard 100 for the patient. The body 112 of each bite block 102, 104 is designed to prevent the bite guard 100 from collapsing when experiencing axial stress exerted from the back molar teeth, wherein the body 112 may have a solid construction, or alternatively could be hollow with internal supporting struts (not shown).

The upper surface 114 is configured to engage the upper teeth of a patient when the bite guard 100 is positioned in the oral cavity, and may include a plurality of raised ridges 120 configured to provide a grip surface to prevent sliding of the first bite block 102 and the second bite block 104 along the upper teeth of the patient. Correspondingly, the lower surface 116 is configured to engage the lower teeth of a patient when the bite guard 100 is positioned in the oral cavity, and may also include a plurality of raised ridges 120 configured to provide a grip surface to prevent sliding of the first bite block 102 and the second bite block 104 along the lower teeth of the patient. The raised ridges 120 may be designed to engage the cusps of the teeth, thereby improving traction and preventing dislodgement of the bite guard 100 during use.

Each of the first and second bite blocks 102, 104 may include an upper barrier 122 extending upwardly from an inner, medial side 124 of the body 112. The upper barrier 122 may be generally planar and smooth with rounded edges, protecting the tongue from laying over the upper surface 114 and experiencing trauma from the maxillary molars during use. Likewise, each of the first and second bite blocks 102, 104 may include a lower barrier 126 extending downwardly from the medial side 124, wherein the lower barrier 126 may be generally planar and smooth with rounded edges, protecting the tongue from laying over the lower surface 116 and experiencing trauma from the mandibular molars during use. In one non-limiting embodiment, the lower barrier 126 may have a greater height dimension than the upper barrier 122 such that the body 112 is generally wedge-shaped, as best shown in FIG. 17.

With continuing reference to FIGS. 14-21, the bridge member 106 is intended to compartmentalize, depress, and protect the tongue during use of the bite guard 100. In one or more embodiments, the bridge member 106 may have an arched configuration, with a central portion 128 extending upwardly from end portions 130 connected to each bite block 102, 104 at the medial side 124 thereof. The bridge member 106 not only connects the bilateral bite blocks 102, 104, but also acts as a stabilizing structure. In one or more embodiments, the bridge member 106 may include raised portions 132 on a superior side 134 thereof, which may increase the rigidity of the bridge member 106 when under flexion and torsional forces. As best shown in FIG. 21, an inferior side 136 of the bridge member 106 may include indicia 138 to provide orientation information regarding the bite guard 100 for the user. In the non-limiting example illustrated, a “B” is used to denote the bottom of the bite guard 100 and the arrow provides clarity as to the direction of insertion of the bite guard 100.

The upper barriers 122 and the superior side 134 of the bridge member 106 define an opening 140 which provides access into the patient’s oral cavity for placement of tubes, scopes, and other equipment, whereas the lower barriers 126 and the inferior side 136 of the bridge member 106 define a comfortable, safe cavity 142 for the patient’s tongue. The opening 140 creates a guiding pathway for easy and reliable insertion of an endotracheal tube or other catheter, scope, or medical equipment into the oral cavity, facilitating safe passage and later adjustment of such devices without causing damage to the patient’s oral structures while allowing wide angle visibility for medical professionals during these procedures. The width of the opening 140 also allows flexibility in positioning and adjusting tubes, scopes, and the like within the patient's mouth.

In one or more embodiments, the bite guard 100 may be constructed from thermoplastic elastomers, plastics, silicones and any other similar bio-compatible materials, and may be constructed to be disposable or reusable and sterilizable. The material used for the bite guard 100 should be strong, rigid, and resilient enough to maintain structural integrity under the biting force of the patient and absorb the shock thereof, yet soft and compliant enough so as to be comfortable to wear and not cause damage to the gums, teeth, and oral cavity. The bite guard 100 may have a scalable design, may be available in a range of different sizes (adult and pediatric patient populations), or a have custom design for each patient. In one or more embodiments, the bite guard 100 is symmetric about a central longitudinal axis 144.

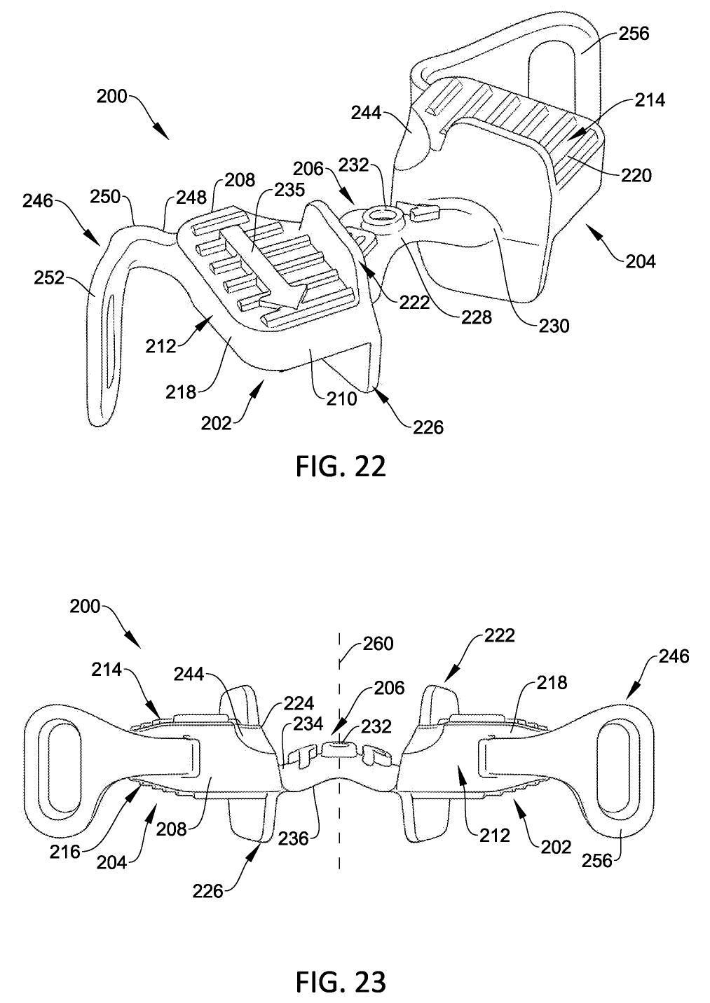

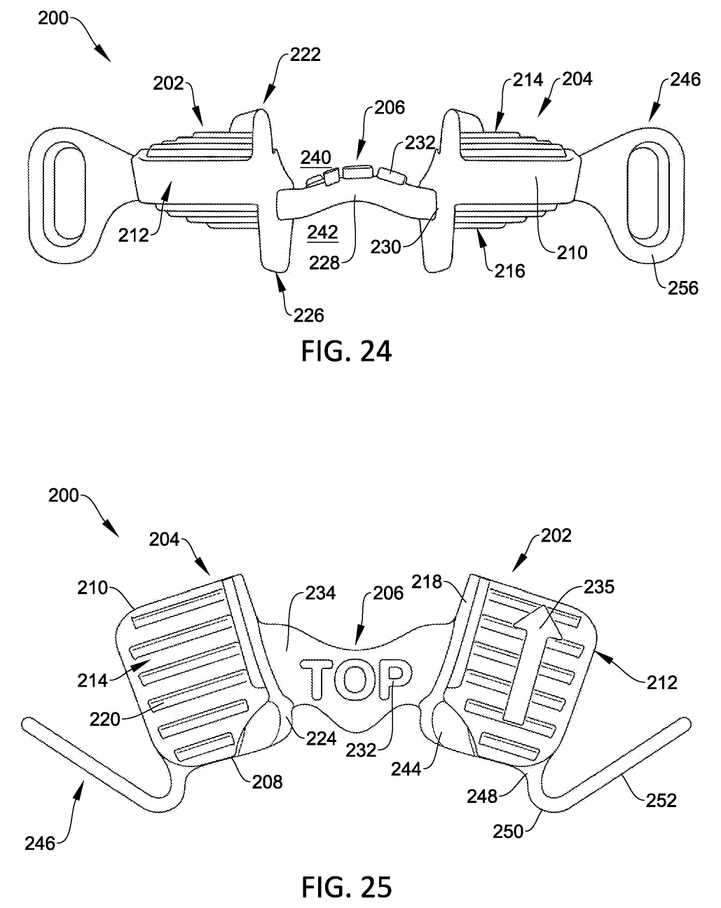

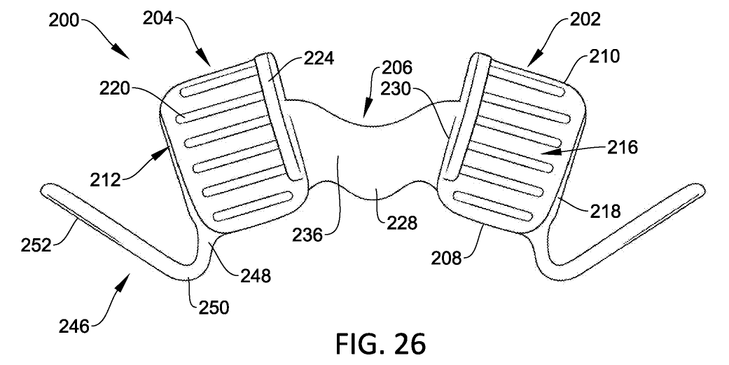

Referring now to FIGS. 22-29, various views of another bite guard 200 according to one or more embodiments are illustrated. It is understood that the features shown and described herein for bite guard 200 may be applicable to bite guards 10, 100 and vice versa.

As with bite guards 10, 100, bite guard 200 is configured for insertion into an oral cavity of a patient suffering from certain medical conditions or undergoing various medical procedures. The bite guard 200 may be designed to be securely retained against the upper teeth and the lower teeth to prevent accidental displacement during intubation or other procedures. This secure retention ensures that proper positioning of the bite guard 200 is maintained so that it can properly protect the patient’s teeth, tongue, and other oral structures. According to one or more embodiments, the bite guard 200 maintains the patient’s mouth in an open position under heavy biting forces, providing continued access to the oral cavity and protecting the oral structures from injury during use.

The bite guard 200 may have a bilateral configuration including a first bite block 202 spaced from a second bite block 204, wherein the first bite block 202 and the second bite block 204 are joined by a bridge member 206. In one or more embodiments, the first bite block 202, the second bite block 204, and the bridge member 206 are integrally formed, although other constructions are also contemplated. As illustrated, the first bite block 202 and the second bite block 204 may be oriented such that a distance between the anterior ends 208 of the first and second bite blocks 202, 204 is smaller than a distance between the posterior ends 210 of the first and second bite blocks 202, 204. Such a configuration corresponds to an expansion of a width of the lower jawbone in the anterior to posterior direction, providing an ergonomic and comfortable fit of the bite guard 200 in a patient’s oral cavity.

In one or more embodiments, the first bite block 202 and the second bite block 204 may each have a generally rectangular body 212 with an upper surface 214 and a lower surface 216 surrounded by an outer surface 218. The outer surfaces 218 of the first bite block 202 and the second bite block 204 may be generally smooth and rounded so as to facilitate comfort for the patient as the bite guard 200 engages parts of the oral cavity during use. The body 212 of each of the first and second bite blocks 202, 204 may each have a thicker anterior end 208 compared with the posterior end 210, corresponding to a decreasing distance between the upper and lower teeth of a patient in an anterior to posterior direction, again facilitating an ergonomic and comfortable fit of the bite guard 200 for the patient. The body 212 of each bite block 202, 204 is designed to prevent the bite guard 200 from collapsing when experiencing axial stress exerted from the back molar teeth, wherein the body 212 may have a solid construction, or alternatively could be hollow with internal supporting struts (not shown).

The upper surface 214 is configured to engage the upper teeth of a patient when the bite guard 200 is positioned in the oral cavity, and may include a plurality of raised ridges 220 configured to provide a grip surface to prevent sliding of the first bite block 202 and the second bite block 204 along the upper teeth of the patient. Correspondingly, the lower surface 216 is configured to engage the lower teeth of a patient when the bite guard 200 is positioned in the oral cavity, and may also include a plurality of raised ridges 220 configured to provide a grip surface to prevent sliding of the first bite block 202 and the second bite block 204 along the lower teeth of the patient. The raised ridges 220 may be designed to engage the cusps of the teeth, thereby improving traction and preventing dislodgement of the bite guard 200 during use.

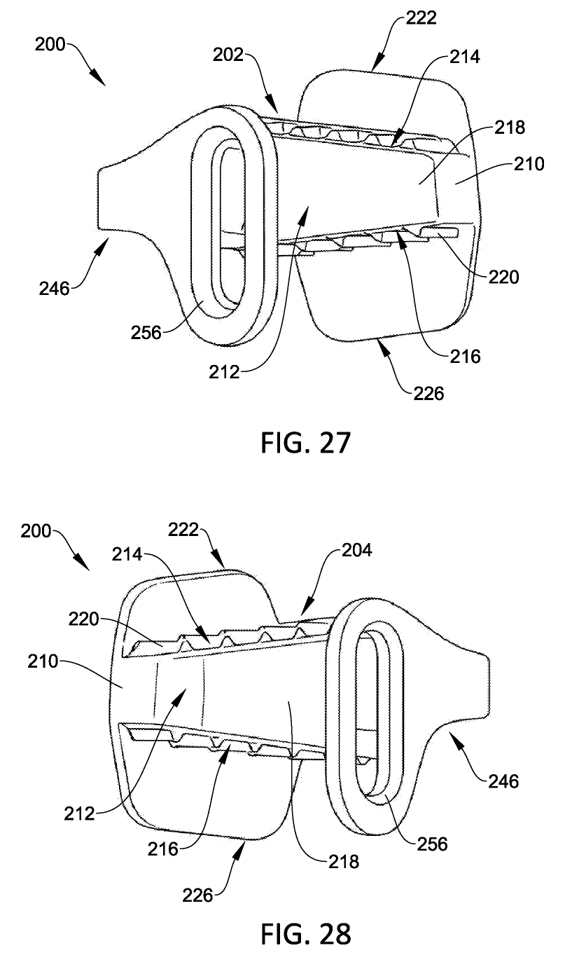

Each of the first and second bite blocks 202, 204 may include an upper barrier 222 extending upwardly from an inner, medial side 224 of the body 212. The upper barrier 222 may be generally planar and smooth with rounded edges, protecting the tongue from laying over the upper surface 214 and experiencing trauma from the maxillary molars during use. Likewise, each of the first and second bite blocks 202, 204 may include a lower barrier 226 extending downwardly from the medial side 224, wherein the lower barrier 226 may be generally planar and smooth with rounded edges, protecting the tongue from laying over the lower surface 216 and experiencing trauma from the mandibular molars during use. In one non-limiting embodiment, the lower barrier 226 may have a greater height dimension than the upper barrier 222 such that the body 212 is generally wedge-shaped, as best shown in FIGS. 27-28.

With continuing reference to FIGS. 22-29, the bridge member 206 is intended to compartmentalize, depress, and protect the tongue during use of the bite guard 200. In one or more embodiments, the bridge member 206 may have an arched configuration, with a central portion 228 extending upwardly from end portions 230 connected to each bite block 202, 204 at the medial sides 224 thereof. The bridge member 206 not only connects the bilateral bite blocks 202, 204, but also acts as a stabilizing structure. In one or more embodiments, the bridge member 206 may include indicia 232 on a superior side 234 thereof to provide orientation information regarding the bite guard 200 for the user. In the non-limiting example illustrated, “TOP” is used as the indicia 232 to denote the top of the bite guard 200. In one or more embodiments, the upper surface 214 of at least one of the bite blocks 202, 204 may include indicia 232 such as an arrow 235 (FIGS. 22 and 25) which may provide clarity as to the direction of insertion of the bite guard 200.

The upper barriers 222 and the superior side 234 of the bridge member 206 define an opening 240 which provides access into the patient’s oral cavity for placement of tubes, scopes, and other equipment, whereas the lower barriers 226 and the inferior side 236 of the bridge member 206 define a comfortable, safe cavity 242 for the patient’s tongue. The opening 240 creates a guiding pathway for easy and reliable insertion of an endotracheal tube or other catheter, scope, or medical equipment into the oral cavity, facilitating safe passage and later adjustment of such devices without causing damage to the patient’s oral structures while allowing wide angle visibility for medical professionals during these procedures. The width of the opening 240 also allows flexibility in positioning and adjusting tubes, scopes, and the like within the patient's mouth. As best shown in FIGS. 22-23, in one or more embodiments, at least one bite block 202, 204 may include a recessed portion 244 of the upper surface 214 formed therein at the anterior ends 208 generally adjacent to the upper barriers 222. This recessed portion 244 may be generally curved, and may provide additional clearance for repositioning of tubes or the like on the superior side 234 of the bridge member 206 from side to side in the patient’s oral cavity.

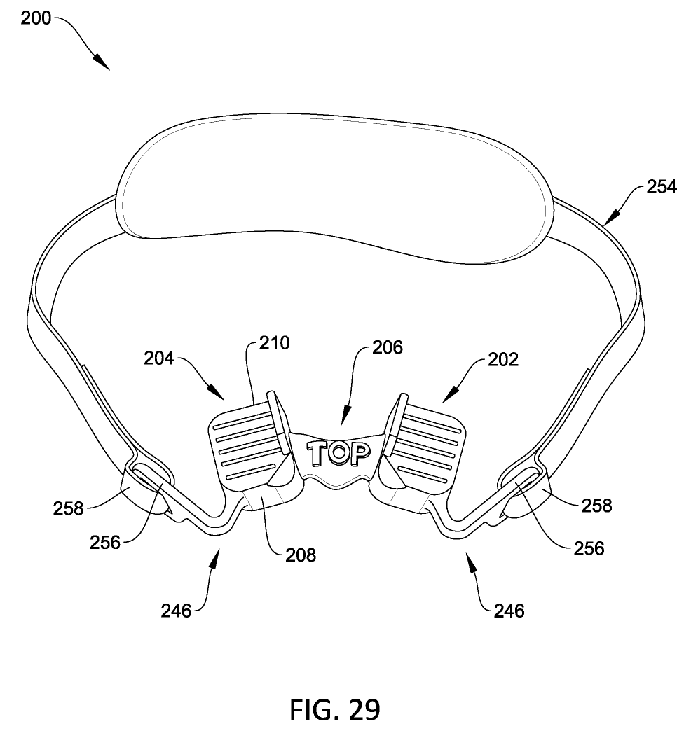

In one or more embodiments, the bite guard 200 may further include arms 246 that extend from the anterior end 208 of each bite block 202, 204, as illustrated in FIGS. 22-29. The arms 246 may be integrally formed with the bite blocks 202, 204 or could be attachable thereto. In the non-limiting embodiment illustrated, each arm 246 may include a proximal section 248 extending outwardly from the anterior end 208, a curved section 250 extending from the proximal section 248, and a distal section 252 extending from the curved section 250. The curved section 250 may curve toward a posterior direction, and the distal section 252 may extend laterally and be configured to rest upon the patient’s cheeks. The arms 246 provide extraoral stabilization and may include an anchoring point for a strap 254 (FIG. 29) which can be placed around the back of the patient’s head or neck during use of the bite guard 200. In one or more embodiments, the distal section 252 may include an attachment portion 256, such as an aperture as shown, to which the strap 254 can be removably secured. The strap 254 may be adjustable, such as via hook and loop (e.g., VELCRO®) ends 258, but it is understood that the strap 254 is not limited to the configuration depicted herein. The arms 246 and strap 254 may reduce the risk of displacement of the bite guard 200 during procedures by ensuring that the bite guard 200 remains properly seated within the oral cavity, even under involuntary jaw movement or prolonged use. In other embodiments, adhesive could be used to secure the arms 246 to the patient’s face for extraoral stabilization in the absence of or in combination with the strap 254.

In one or more embodiments, the bite guard 200 may be constructed from thermoplastic elastomers, plastics, silicones and any other similar bio-compatible materials, and may be constructed to be disposable or reusable and sterilizable. The material used for the bite guard 200 should be strong, rigid, and resilient enough to maintain structural integrity under the biting force of the patient and absorb the shock thereof, yet soft and compliant enough so as to be comfortable to wear and not cause damage to the gums, teeth, and oral cavity. The bite guard 200 may have a scalable design, may be available in a range of different sizes (adult and pediatric patient populations), or a have custom design for each patient. In one or more embodiments, the bite guard 200 is symmetric about a central longitudinal axis 260.

Embodiments of the bite guard 10, 100, 200 shown and described herein function to protect the oral cavity of the patient by creating a supported opening to prevent contact between the teeth and tongue, shielding oral structures from contact and potential injury by tubes, instruments, and the like, and maintaining unobstructed access to the oral cavity. The bite guards 10, 100, 200 shown and described herein may be used in hospital settings, ICU units, operating rooms, and inpatient and outpatient clinics, and have many use cases across multiple disciplines of medicine. In one or more embodiments, the bite guard 10, 100, 200 may be utilized in anesthesia cases to protect the patient’s dentition during or after intubation. Another target population are patients undergoing trans-esophageal procedures, inclusive of many existing and future procedures in the disciplines of gastrointestinal surgery, oral and maxillofacial surgery, pulmonary surgery, and others. Other populations which can greatly benefit from the bite guard 10, 100, 200 are patients experiencing neurogenic bruxism, or patients with sleep apnea. Of course, it is understood that the bite guards 10, 100, 200 are not limited to use in these scenarios, disorders, or diseases, but rather the bite guards 10, 100, 200 are contemplated for use with any patient that is undergoing intubation/assisted ventilation in both inpatient and outpatient clinics or hospitals. Other current and future use cases in both the adult and pediatric patient populations are fully contemplated.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A bite guard configured for insertion into an oral cavity of a patient, the bite guard comprising:
a first bite block and a second bite block spaced apart laterally, the first bite block and the second bite block each including an upper surface configured to engage upper teeth of the patient and a lower surface configured to engage lower teeth of the patient, each of the first and second bite blocks having an anterior end and a posterior end;
a bridge member joining the first bite block and the second bite block at medial sides thereof;
upper barriers extending upwardly from the medial sides of the first and second bite blocks; and
lower barriers extending downwardly from the medial sides of the first and second bite blocks,
wherein the upper barriers and a superior side of the bridge member define an opening providing access to the oral cavity, and the lower barriers and an inferior side of the bridge member define a cavity configured to receive a tongue of the patient.

2. The bite guard of claim 1, wherein a distance between the anterior ends is smaller than a distance between the posterior ends.

3. The bite guard of claim 1, wherein the anterior end of each of the first and second bite blocks is thicker than the posterior end of each of the first and second bite blocks.

4. The bite guard of claim 1, wherein at least one of the first and second bite blocks includes a recessed portion formed in the upper surface adjacent to the anterior end and the upper barriers.

5. The bite guard of claim 1, wherein the first bite block and the second bite block each have a generally rectangular body surrounded by a smooth, rounded outer surface.

6. The bite guard of claim 1, wherein the upper surface and the lower surface of each of the first and second bite blocks include a plurality of raised ridges.

7. The bite guard of claim 1, wherein the upper barriers and the lower barriers each have a generally planar configuration with rounded edges.

8. The bite guard of claim 1, wherein the lower barriers have a greater height dimension than the upper barriers.

9. The bite guard of claim 1, wherein the bridge member has an arched configuration including a central portion extending upwardly relative to end portions connected to the first and second bite blocks.

10. The bite guard of claim 1, wherein at least one of the bridge member, the first bite block, and the second bite block includes orientation indicia thereon.

11. The bite guard of claim 1, further comprising arms extending from the anterior ends of the first and second bite blocks.

12. The bite guard of claim 11, wherein the arms each include a proximal section, a curved section extending from the proximal section in a posterior direction, and a distal section extending laterally from the curved section.

13. The bite guard of claim 11, wherein the arms each include an attachment portion configured to receive a retaining strap.

14. A bite guard configured for insertion into an oral cavity of a patient, the bite guard comprising:
a first bite block and a second bite block spaced apart laterally, the first bite block and the second bite block each including an upper surface configured to engage upper teeth of the patient and a lower surface configured to engage lower teeth of the patient, each of the first and second bite blocks having an anterior end and a posterior end;
a bridge member joining the first bite block and the second bite block at medial sides thereof;
upper barriers extending upwardly from the medial sides of the first and second bite blocks, and lower barriers extending downwardly from the medial sides of the first and second bite blocks, wherein the upper barriers and the bridge member define an opening providing access to the oral cavity, and the lower barriers and the bridge member define a cavity configured to receive a tongue of the patient;
arms extending from the anterior ends of the first and second bite blocks, each arm including an attachment portion; and
a strap removably secured to the attachment portions, wherein the arms and the strap are configured to provide extraoral stabilization of the bite guard.

15. The bite guard of claim 14, wherein the strap is adjustable.

16. A bite guard configured for insertion into an oral cavity of a patient, the bite guard comprising:
a generally U-shaped upper member having an upper surface configured to engage upper teeth of the patient, the upper member having spaced upper side portions with an upper anterior end portion therebetween;
a generally U-shaped lower member spaced from the upper member and having a lower surface configured to engage lower teeth of the patient, the lower member having spaced lower side portions with a lower anterior end portion therebetween; and
a support structure joining the upper side portions with the lower side portions, the support structure including a first support member and a second support member, wherein an opening is defined between the upper anterior end portion and the lower anterior end portion and extending through the bite guard between the first support member and the second support member for providing access into the oral cavity.

17. The bite guard of claim 16, wherein the upper member includes an upper lip barrier extending upwardly from the upper anterior end portion, and the lower member includes a lower lip barrier extending downwardly from the lower anterior end portion.

18. The bite guard of claim 17, wherein the upper lip barrier has a generally curved shape symmetric about a central longitudinal axis and includes an indentation configured to accommodate a maxillary frenum of the patient.

19. The bite guard of claim 17, wherein the lower member further includes a ledge extending downwardly from a posterior edge of the lower anterior end portion, the ledge configured to latch onto mandibular incisors of the patient.

20. The bite guard of claim 16, wherein the upper member includes upper medial lips extending upwardly from inner edges of side portions of the upper member, and wherein the lower member includes lower medial lips extending downwardly from inner edges of side portions of the lower member, the upper medial lips and the lower medial lips configured to inhibit a tongue of the patient from contacting the upper surface and the lower surface, respectively.
